(19) United States
(12) Patent Application Publication
(10) Pub. No.: US 2022/0265340 A1
(43) Pub. Date: Aug. 25, 2022

(54) ELECTROSURGICAL HANDHELD DEVICE, AND CONTACT BODY FOR AN ELECTROSURGICAL HANDHELD DEVICE

(71) Applicant: Olympus Winter & Ibe GmbH, Hamburg (DE)

(72) Inventors: Andreas OFFT, Reinbek (DE); Christian BROCKMANN, Hollenstedt (DE); Nils TORKUHL, Gross Vollstedt (DE); Dorian DRECHSLER, Hamburg (DE)

(73) Assignee: Olympus Winter & Ibe GmbH, Hamburg (DE)

(21) Appl. No.: 17/677,044

(22) Filed: Feb. 22, 2022

(30) Foreign Application Priority Data
Feb. 22, 2021 (DE) 10 2021 104 119.8

(51) Int. Cl. A61B 18/14

(52) U.S. Cl. CPC A61B 18/149; A61B 2018/0091

(57) ABSTRACT

In known electrosurgical handheld devices, a strengthening tube is firmly connected to a working element or a main body. Before the strengthening tube is connected to the main body, a contact body has to be pushed over the strengthening tube. For this purpose, the contact body has a corresponding bore. When the contact body is replaced for maintenance reasons or because of defects, the strengthening tube has to be removed with difficulty from the main body in order to be able to withdraw the contact body from same. The invention makes available an electrosurgical handheld device and a contact body which is able to be manipulated and maintained in a particularly simple and also time-efficient manner. This is achieved by the fact that a contact body for an electrosurgical handheld device has a slit parallel to a continuous bore and also parallel to a longitudinal axis of the handheld device.

### DESCRIPTION

The invention relates to a contact body for electrosurgical handheld devices according to the preamble of claim 1. The invention also relates to an electrosurgical handheld device according to claim 12.

Electrosurgical handheld devices, for example resectoscopes, are used mainly for endoscopic applications in urology and gynecology, there preferably for treatment in the region of the bladder, the uterus or the prostate. However, the field of use of these instruments is not limited to these regions of the human body and instead also includes the treatment of further organs in the lower part of the human abdomen.

The instruments of the kind described here, for example resectoscopes, have a working element as standard. To treat the diseased tissue, the resectoscope is inserted with an elongate shaft through an opening into the body of the patient. Various medical instruments for treating and/or examining the patient can be arranged in this shaft tube. For example, an electrode to which radiofrequency alternating current can be applied and which is positioned at a distal end of an electrode carrier, can be inserted into the shaft tube for radiofrequency surgery. For treatments that are to be performed on the patient, for example the cutting of diseased tissue, the electrode carrier with the electrode is arranged on the resectoscope in such way as to be movable relative to the shaft tube and along a shaft axis. The electrode or the tool is assigned to the distal end of the shaft tube.

The electrode carrier is furthermore coupled with its proximal end to the working element, by which it can be moved along the shaft axis. The cutting movement of the electrode is permitted in this way. The working element is usually coupled releasably to the shaft tube. It has a movably mounted contact body, which is also designated as a slide. On this contact body, the electrode carrier can be mechanically and releasably coupled to at least one electrical contact. By way of this mechanical connection, the electrode carrier or the electrode can also be supplied with electrical energy. For this purpose, the contact body has at least one opening into which at least one electrical contact of the electrode carrier can be guided for the releasable connection. This opening or this blind hole or this bore is designed in such a way that the RF voltage can be applied via an adjoining plug socket. For this purpose, a plug is usually fitted into the socket and is in turn connectable to an RF generator via a line or a cable.

The working element is actuated or longitudinally displaced by an operator. For this purpose, the working element is assigned a grip unit with a first gripping means and a second gripping means. To actuate the working element, the operator grasps the first gripping means, and also a second gripping means that can have a finger unit or a thumb ring. The first gripping means can be fastened to a stationary main body of the working element. The second gripping means can be fastened to the contact body.

The movement of the working element takes place counter to a spring tension of a spring, which in the working element of the type in question is designed usually as a leaf spring or as a leg spring. One end of this spring is fastened to the contact body or the slide, and the other end is fastened to an end body or an optical guide plate of a strengthening tube. The nature of the springs or the type of actuation of this spring mechanism depends on whether the working element is an active or passive working element. Whereas the spring in the case of an active working element is designed as a compression spring, in the case of a passive working element it is designed as a tension spring.

The cutting by the electrode is usually effected by a pulling-back movement of the working element. In the case of the active working element, the electrode is for this purpose pulled back (in the proximal direction) against the spring force of the spring. By contrast, in the case of the passive working element, the electrode is first of all pushed forward (in the distal direction) counter to the spring force, in order then to cut through the tissue during the return movement (in the proximal direction) caused by the relaxation of the spring.

An optical unit can also be guided through the shaft of the instruments described here. The rod-like or shaft-like optical unit is guided from the proximal end through a strengthening tube, also called optical guide tube, of the working element into the shaft.

Embodiments are known in which the optical unit is guided as a rod lens system or as an optical fiber through the shaft from a proximal end to the distal end. The distal end of the optical unit is directed directly to the surgical region or the site of action of the electrode. At the proximal end of the optical unit, the operator can observe the treatment through an eyepiece or a camera.

The electrode system is connected to the at least one electrical contact of the contact body, and both are then pushed together into or through the shaft. By actuation of the second gripping means relative to the first gripping means, the contact body together with the electrode carrier can thus be moved to and fro along the shaft axis via the strengthening tube.

In known systems, the strengthening tube is connected fixedly to the working element or the main body at the time of manufacture. At the proximal end of the strengthening tube, the latter is welded to the optical guide plate. Before the strengthening tube is connected to the main body, the contact body has to be pushed over the strengthening tube. For this purpose, the contact body has a corresponding bore parallel to the shaft axis. This bore is dimensioned such that the contact body or the slide can be pushed easily over the strengthening tube.

It is not only the complicated manufacture of the working element that has proven to be particularly disadvantageous, but also the increased effort involved in the repair and replacement of components. Particularly when the contact body is to be replaced for maintenance reasons or because of defects, the strengthening tube has to be removed with difficulty from the main body in order to be able to withdraw the contact body from same.

Proceeding from this, the object of the invention is to make available an electrosurgical handheld device and a contact body which can be manipulated and serviced in a particularly simple and time-efficient manner.

A contact body for achieving this object has the features of claim 1. Accordingly, provision is made that the contact body for an electrosurgical handheld device has a slit parallel to a continuous bore and also parallel to a longitudinal axis of the handheld device. The bore is designed to receive an optical guide. This optical guide can be designed, for example, as a strengthening tube or as a tubular shaft for receiving a rod-like optical unit. The bore extends from an end face of the contact body to an opposite end face, wherein the bore is oriented parallel to a longitudinal axis of the electrosurgical handheld device.

According to the invention, the slit is designed in such a way that the contact body can be plugged over the tubular optical guide, wherein the optical guide is moved through the slit into the bore. Of course, it is equally conceivable too that the tubular optical guide can be pressed through the slit into the bore. By virtue of the slit, the contact body can thus also be fastened to a handheld device, or a working element of a handheld device, that has been at least almost finally assembled. Equally, the contact body can be released from the optical guide for maintenance purposes, specifically without the handheld device needing to be disassembled to any great extent. By virtue of the contact body according to the invention, production and maintenance can therefore be particularly easy and time-efficient.

In particular, the invention can further provide that the slit extends through the contact body from an outer wall of the contact body as far as the bore, wherein the tubular optical guide can be guided through the slit into the bore. The slit and the bore thus together form a recess in the contact body. Ultimately, the slit constitutes an extension of the interior of the bore. Through this extension, the optical guide can be easily inserted in the bore and also removed again. Provided that the slit-like design of the extension is not too wide, nothing changes as regards the sliding connection between the optical guide and an inner wall of the bore. Rather, the handling of the handheld device is unaffected by this slit.

Preferably, it is moreover conceivable that the slit has two side walls, which are parallel or enclose an angle, or that the side walls have a triangular cross section, wherein two corners of the side walls lie directly opposite and parallel to each other. These designs of the side walls ensure that the optical guide can be inserted into the bore and also removed from the latter again. It additionally ensures that the optical guide does not accidentally slip out of the bore through the slit.

Preferably, it is moreover conceivable that a cross section of the contact body has an annular, preferably circular or oval shape, wherein the slit constitutes an opening in the shape. With this annular design, it is conceivable that the open ring ends that form the slit are reversibly deformable, in order to be at least temporarily moved apart from each other during reception of the optical guide, such that the optical guide can be inserted into the bore. As soon as the optical guide is placed in the bore, the contact body can readopt its original shape. It is equally conceivable that the contact body does not deform, and instead a cross section of the tubular optical guide is at least temporarily modifiable in order to be guided through the slit into the bore.

In a further advantageous illustrative embodiment of the invention, provision can be made that a plane extending parallel and centrally between the side walls intersects a central axis of the bore. By means of this relative orientation of the side walls and of the bore, the optical guide can be guided in a particularly simple way into the bore. It is additionally conceivable that the aforementioned plane is slightly offset from the central axis. This can facilitate the insertion and removal of the optical guide into and from the bore.

In a particularly advantageous embodiment of the invention, provision is made that a width of the slit, i.e. a distance between the side walls thereof, is less than the diameter of the bore. The bore can preferably have a diameter of 3 mm to 6 mm, preferably 4 mm to 5 mm, in particular 4.6 mm, and the slit can have a width of 2 mm to 5 mm, preferably 3 mm to 4 mm, in particular 3.5 mm. The diameter of the bore is always slightly greater than the diameter of the tubular optical guide.

A ratio between a width of the slit, in particular a minimum spacing of the side walls of the slit, and a diameter of the bore of 0.6 to 0.9, preferably 0.7 to 0.8, in particular 0.76, has proven particularly preferable. This ratio of the width and of the diameter is particularly preferable for easy insertion of the optical guide and also for a sufficient sliding resistance of the contact body on the optical guide. If the ratio is too small, there is in particular the risk of the structural parts plastically deforming. If the ratio is too great, the guiding function is no longer ensured. The described values apply for PTFE and may differ for other materials.

The contact body of the surgical handheld device described here can also be designed as a slide of an active or passive resectoscope. It has been found that plastic, in particular PTFE, is particularly advantageous for the contact body on account of the material properties, for example lower sliding resistance, high electrical resistance, smooth surface and good processability. However, it is equally conceivable too that the contact body is made of another fluoropolymer, PFA for example. PEEK has also proven advantageous.

It is further conceivable according to the invention that the contact body has at least one receptacle for a contact of the electrode instrument, and this receptacle is connectable to at least one plug socket, in particular that a plug socket is integrated in the contact body. If the surgical handheld device is an electrode instrument with only one electrical contact, the contact body likewise has only one corresponding receptacle. However, if the electrode instrument has two contacts, for example an active contact and a return contact, then the contact body can accordingly have two receptacles for the electrical contacting. These receptacles are likewise arranged parallel to the bore in the contact body and can be designed like a blond hole or extend all the way through the entire body.

An electrosurgical handheld device for achieving the object mentioned at the outset has the features of claim 12. Accordingly, provision is made that the handheld device, which can preferably be a resectoscope with an active or passive working element, has an electrode instrument which at a distal end has an electrode and at a proximal end has at least one electrical contact. Moreover, the handheld device has a grip unit with a first gripping means and a second gripping means. In addition, the instrument has a tubular shaft which is coupled with a proximal end to the first gripping means, and an optical guide for receiving an optical unit. This optical guide can be guided through a contact body, wherein the second gripping means and a spring are also fastened to this contact body, and has at least one receptacle for an electrical contact of the electrode instrument. According to the invention, this contact body is designed according to at least one of the preceding claims.

A preferred illustrative embodiment of the invention is explained in more detail below with reference to the drawing, in which:

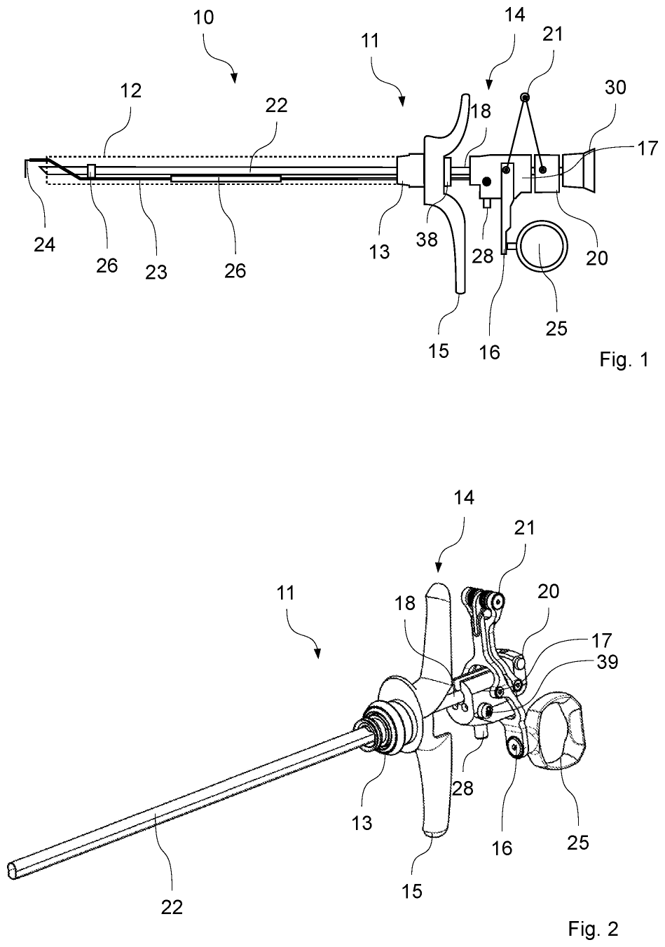

FIG. 1 shows a schematic view of a resectoscope,

FIG. 2 shows a schematic view of a working element,

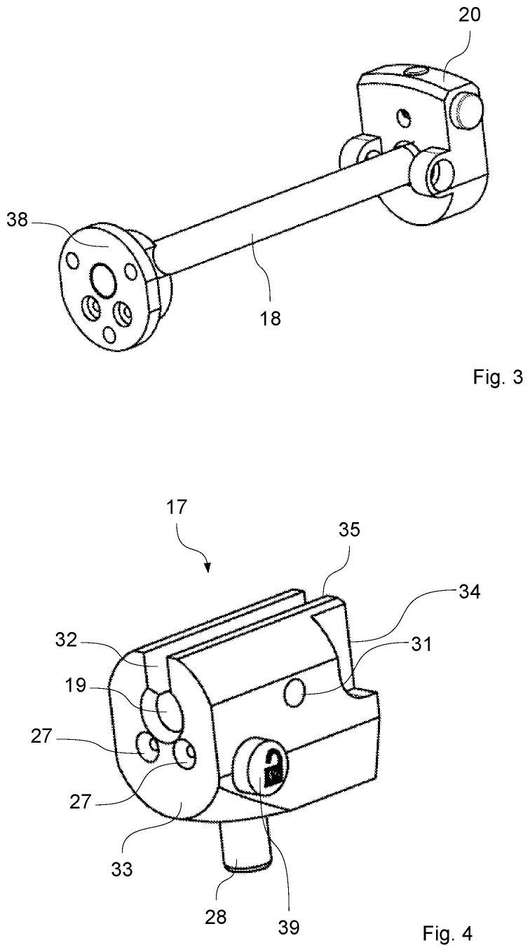

FIG. 3 shows a perspective view of an optical guide,

FIG. 4 shows a perspective view of a contact body, and

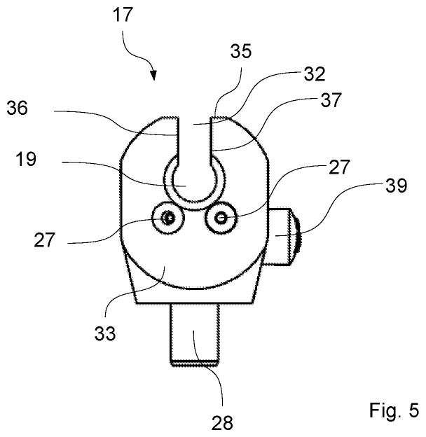

FIG. 5 shows a view of an end face of the contact body according to FIG. 4.

A possible illustrative embodiment of an electrosurgical handheld device, namely a resectoscope 10, is depicted highly schematically in FIG. 1. The resectoscope 10 has a working element 11 (see also FIG. 2) on which an elongate, tubular shaft 12 can be fastened. This shaft 12 is indicated by hatching in FIG. 1 and is fastened with a proximal end to a main body 13 of the working element 11.

The working element 11 has, in addition to the main body 13, a grip unit 14. This grip unit 14 has a first gripping means 15 and a second gripping means 16. While the first gripping means 15 is arranged fixedly on the main body 13, the second gripping means 16 is assigned to a contact body 17 in the illustrative embodiment of the working element 11 shown here. It is conceivable that the second gripping means 16 is screwed firmly on the contact body 17. For this purpose, the contact body 17 has a corresponding bore 31 in a wall.

The contact body 17 is guided slidingly on a tubular optical guide 18. For this purpose, the contact body 17 has a bore 19 whose diameter is slightly greater than a diameter of the optical guide 18. Since the contact body 17 can move to and fro on the optical guide 18 along a longitudinal direction of the resectoscope 10 or a longitudinal axis of the shaft 12, the contact body 17 is also designated as a slide.

While the optical guide 18 is connected with a distal end to the main body 13 or an inner tube 22 via an adapter 38 (FIG. 3), an optical guide plate 20 is fastened at a proximal end of the optical guide 18. The tubular optical guide 18 extends through the optical guide pate 20, such that the optical guide 18 is accessible from the proximal direction.

The second gripping means 16 and the contact body 17 are connected to the optical guide plate 20 via a spring element 21. This spring element 21 can be a tension spring.

Starting from the main body 13, an inner tube 22 extends in the distal direction. This inner tube 22 can also extend in the proximal direction through the main body 13 and be connected to the optical guide 18. It is equally conceivable that the inner tube 22 and the optical guide 18 are formed in one piece, or that the optical guide 18 extends distally through the main body 13.

An electrode instrument 23 extends parallel to the inner tube 22. This electrode instrument 23 is guided through the main body 13 and with at least one proximal contact is mechanically and releasably coupled in a receptacle 27 to the contact body 17. In the contact body 17, a latching mechanism can be provided which can be released and fixed via a button 39 (FIGS. 4 and 5). The latching mechanism locks the at least one proximal end or the contact of the electrode instrument 23 in the contact body 17. The button 39 or the latching mechanism can be spring-preloaded and can be easily operated with one finger.

At a distal end, the electrode instrument 23 has an electrode 24. An electrical RF voltage can be applied to this electrode 24. The diseased tissue can be manipulated or cut by means of a plasma that forms at the electrode 24. For this purpose, the operator moves the second gripping means 16, having a thumb ring 25, relative to the first gripping means 15. For stabilizing the electrode instrument 23, the latter can be guided on the inner tube 22 by guides 26.

For applying RF voltage to the electrode 24, the receptacle 27 of the proximal contact of the electrode instrument 23 can be electrically contacted. For this purpose, the contact body 17 has at least one plug socket 28 (FIGS. 4 and 5). This plug socket 28 is in electrical contact with at least one part of an inner wall of the receptacle 27. By way of a plug (not shown here), the contact body 17 can thus be connected by a cable to an RF generator.

For performing the intervention, a rod-like optical unit 29 is guided through the inner tube 22 or optical guide 18. A distal end (not visible here) of this optical unit 29 is directed in the direction of the electrode 24, so that the operator has a view of the manipulation of the tissue. This optical unit 29 can be a rod lens system or an optical fiber. As is shown in FIG. 1, an eyepiece 30 or a camera is located at the proximal end of the optical unit 29.

In the manufacture of the working element 11, the assembly of the contact body 17 proves particularly awkward. It was hitherto the case that the optical guide plate 20 was first welded to the optical guide 18, then the contact body 17 was plugged onto the optical guide 18, and then the optical guide 18 was firmly connected by the distal end to the main body 13 or the inner tube 22. For replacement or for maintenance of the contact body 17, these steps had to be repeated in reverse.

The contact body 17 shown here has a slit 32 (FIG. 4). This slit 32 extends parallel to the bore 19 from an end face 33 to the opposite end face 34 of the contact body 17. The slit 32 is designed in such a way that it extends from an outer wall 35 as far as the bore 19 (FIG. 5). The interior of the bore 19 is thus extended.

In the illustrative embodiment of the slit 32 shown in FIG. 5, it has two parallel side walls 36, 37. The distance between these two side walls 36, 37, i.e. the width of the slit 32, is less than the diameter of the bore 19. Provision is made in particular that a ratio between the width of the slit 32 and the diameter of the bore 19 is 0.6-0.9, preferably 0.7-0.8, in particular 0.76.

By virtue of the extension of the bore 19 through the slit 32, it is possible to clip the contact body 17 onto the optical guide 18. For this purpose, the tubular optical guide 18 is guided through the slit 32 into the bore 19. It is conceivable here that an external diameter of the optical guide 18 or the distance between the two side walls 36, 37 deforms for a short time and in a reversible manner. After the assembly of the contact body 17, the gripping means 16, the spring element 21 and the electrode instrument 23 can then be connected to the contact body 17. If necessary, the aforementioned components of the working element 11 can be quickly and easily released from the contact body 17, in order then to withdraw the contact body 17 from the optical guide 18 for maintenance purposes.

### LIST OF REFERENCE SIGNS

10 resectoscope

11 working element

12 shaft

13 main body

14 grip unit

15 first gripping means

16 second gripping means

17 contact body

18 optical guide

19 bore

20 optical guide plate

21 spring element

22 inner tube

23 electrode instrument

24 electrode

25 thumb ring

26 guide

27 receptacle

28 plug socket

29 optical unit

30 eyepiece

31 bore

32 slit

33 end face

34 end face

35 outer wall

36 side wall

37 side wall

38 adapter

39 button

1. A contact body for an electrosurgical handheld device, a resectoscope, for receiving an optical guide, for fastening a grip unit, and for coupling at least one electrical contact of an electrode instrument of the handheld device, wherein the contact body has a slit parallel to a continuous bore for receiving the optical guide and also parallel to a longitudinal axis of the handheld device.

2. The contact body for an electrosurgical handheld device as claimed in claim 1, wherein the slit extends from an outer wall of the contact body as far as the bore, wherein the tubular optical guide can be guided through the slit into the bore.

3. The contact body for an electrosurgical handheld device as claimed in claim 1, wherein the slit has two side walls, which are parallel or enclose an angle, or in that the side walls have a triangular cross section, wherein two corners of the side walls lie directly opposite and parallel to each other.

4. The contact body for an electrosurgical handheld device as claimed in claim 1, wherein a cross section of the contact body has an annular, preferably circular or oval shape, wherein the slit constitutes an opening in the shape.

5. The contact body for an electrosurgical handheld device as claimed in claim 1, wherein a plane extending parallel and centrally between the side walls intersects a central axis of the bore.

6. The contact body for an electrosurgical handheld device as claimed in claim 1, wherein a width of the slit is less than the diameter of the bore.

7. The contact body for an electrosurgical handheld device as claimed in claim 1, wherein the bore has a diameter of 3 mm to 6 mm, and the slit has a width of 3 mm to 4 5 mm, wherein the diameter is slightly greater than a diameter of the tubular optical guide.

8. The contact body for an electrosurgical handheld device as claimed in claim 1, wherein a ratio between a width of the slit, and a diameter of the bore is 0.6 to 0.9.

9. The contact body for an electrosurgical handheld device as claimed in claim 1, wherein the contact body is a slide for an active or passive resectoscope.

10. The contact body for an electrosurgical handheld device as claimed in claim 1, wherein the contact body is made of plastic.

11. The contact body for an electrosurgical handheld device as claimed in claim 1, wherein the contact body has at least one receptacle for a contact of the electrode instrument, and this receptacle is connected to at least one plug socket, in particular in that a plug socket is integrated in the contact body.

12. An electrosurgical handheld device, with an electrode instrument which at a distal end has an electrode and at a proximal end has at least one electrical contact with a grip unit consisting of a first gripping means and a second gripping means, with a tubular shaft which is coupled with a proximal end to the first gripping means, with an optical guide for receiving an optical unit, and a contact body through which the optical guide can be guided, the second gripping means can be fastened, and in which the at least one electrical contact of the electrode instrument can be latched and/or electrically contacted, wherein a contact body as claimed in claim 1.
